(19) United States
(12) Patent Application Publication
(10) Pub. No.: US 2007/0258849 A1
(43) Pub. Date: Nov. 8, 2007

(54) Methods, systems, and apparatus for creating and transmitting aroma delivery instructions and recording over the internet

(76) Inventor: Carl Ernest Kent, Minneapolis, MN (US)

Correspondence Address: Carl E. Kent, P.O. Box 18431, Minneapolis, MN, 55418, US

(21) Appl. No.: 11/799,601

(22) Filed: May 3, 2007

Related U.S. Application Data
(60) Provisional application No. 60/798,212, filed on May 6, 2006.

(51) Int. Cl. A61L 9/00

(52) U.S. Cl. 422/5; 422/123

(57) ABSTRACT

A scent formulating and scent diffusing apparatus for synchronously mixing one or more scents, spraying one or more scents and recording one or more digital images upon electronic signal demand, with said signaling mechanism either located directly within a common commercially available microprocessor, or over a network of distributed computers such as the world wide web, commonly referred to as the Internet, a computer implemented scent formulation method, scent diffusion system, and a data management method and interactive computer program to synchronize, onto recording subject, the delivery of formulated scents and the recording of computer generated images, graphic art, line art, and text images. In addition, the methods, systems, apparatus, computer programs and compositions of the present invention provide the ability to create, access, manipulate, categorize, store and forward said manipulations of a hierarchical database optimally suited for use in custom scent mixing and scent archival. The present invention also provides the ability to mine data from a database containing a plurality of chemical fingerprints. The collective claims and means herein for methods, systems, and apparatus, constitute the present invention of creating and transmitting aroma delivery instructions over the Internet, herein referred to as aroma casting, aroma streaming, or online scent management.

### RELATED APPLICATION(S)

Not applicable.

### FEDERALLY SPONSORED RESEARCH OR DEVELOPMENT

Not applicable.

### SEQUENCE LISTING OF TABLES OR COMPUTER PROGRAMS OR COMPACT DISC

Not applicable.

### BACKGROUND OF THE INVENTION

1. Technical Field

A scent formulating and scent diffusing apparatus and a data management method and interactive computer program for synchronously mixing one or more scents, spraying one or more scents, and recording one or more digital images upon electronic signal demand, with said signaling mechanism either located directly within a common commercially available personal computer, or over a network of computers such as the world wide web, commonly referred to as the Internet, and more particularly, the storage of individual scents in specially formed scent vessels, each capable of being subdivided with compartmentalized walls to hold different scented fragrant oil, and subsequent mixing of said scents in a wide array of precise formulations and spraying of said scents in precise addressable spraying locations determined by the apparatus' interactions with a database of scent formulation data and scent spraying location coordinates. The present invention also provides the ability to mine data from a database containing a plurality of chemical fingerprints.

2. Prior Art References

| | |
|---|---|
| Suggested U.S. Class: | 347/20; 347/95 |
| Suggested Intern'l Class: | B41J 002/015 |
| Suggested Field of Search: | 347/20, 85, 86, 87; 348/552; |
| | 352/85; 137/883 |

### REFERENCES CITED [REFERENCED BY]

U.S. Patent Documents

| | | | |
|---|---|---|---|
| 5477249 | December, 1995 | Hotomi | 347/48. |
| 5515085 | May., 1996 | Hotomi et al. | 347/54. |
| 5724256 | March, 1998 | Lee et al. Assigned to | 700/285. |
| | | International Business | |
| | | Machines | |
| 5949522 | September, 1999 | Manne | 352/85. |
| 5975675 | November, 1999 | Kim | 347/20. |
| 6169595 | January, 2001 | Manne | 352/85. |
| 6323912 | November, 2001 | McIntyre assigned to | 348/552. |
| | | Kodak | |
| 6325475 | December, 2001 | Hayes et al. assigned | 347/2. |
| | | to Microfab | |
| 6371451 | April, 2002 | Choi assigned to Korea | 261/26. |
| 6731165 | April, 2002 | Inst of Science & | 137/883. |
| | | Technology | |
| | | Manne | |

U.S. Pat. No. 5,477,249 for an Apparatus and Method for Forming Images by Jetting Recording Liquid onto an Image Carrier by Applying both Vibrational Energy and Electrostatic Energy issued to Hotomi and U.S. Pat. No. 5,515,085 for an Ink-Jet Type Recorder issued to Hotomi et al. both disclose mixing of ink with perfume. In each of these patents, the addition of perfume can be added to the pigment dispersion of ink. Thus, the constitution of the ink is improved by the addition of perfume. This patent does not disclose methods for mixing scents independent of mixing inks, actuation over the Internet, nor does this patent synchronously deliver both recorded images and scent.

In U.S. Pat. No. 5,724,256 for Computer Controlled Olfactory Mixer and Dispenser for Use in Multimedia Computer Applications issued to Lee et al. and assigned to International Business Machines, Corporation., we are taught that a system using a plurality of storage containers, each suitable for of storing a different odorant, and using an array of metering delivery devices and a moving absorptive porous member, the combination of which mixes odorants and forces the movement of odorant by means of vaporizing said odorant and then pumping air to disperse said odorant into the air. This patent does not disclose methods for mixing scents, actuation over the Internet, nor does this patent synchronously deliver both recorded images and scent.

U.S. Pat. No. 5,949,522 for Multimedia Linked Scent Delivery System issued to Manne, and U.S. Pat. No. 6,169,595 for Multimedia Linked Scent Delivery System issued to Manne, and U.S. Pat. No. 6371,165 for Dynamic Alloy Wire Valve for a Multimedia Linked Scent Delivery System issued to Manne all disclose the use of proportional flow control valves to deliver precise and predictable mixing and spraying of scent in concert with the delivery of digitized images in frame by frame motion pictures or delivery of scent based on fluctuations in music. These patents do not disclose methods for actuation over the Internet, nor do these patents synchronously delivering both recorded images and scent.

U.S. Pat. No. 5,975,675 for a Perfume Spraying Recorder issued to Kim and assigned to SamSung Electronics Co., Ltd., teaches us that a perfume spraying apparatus with a user selected combination of perfumes, can spray perfumes in to the margins and between lines of recorded characters on recording subject. This patent does not disclose methods for mixing scents, actuation over the Internet, nor does this patent synchronously deliver both recorded images and scent.

U.S. Pat. No. 6,323,912 for an Electronic Camera With Microfluidic Recorder That Recordings Scented Images issued to McIntyre and assigned to Eastman Kodak Company, discloses the use of a recorder to spray scent, but there are no methods disclosed to mix scent, there is no database disclosed, and there is no means for Internet based actuation of the apparatus.

In U.S. Pat. No. 6,325,475 for Devices for Presenting Airborne Materials to the Nose issued to Hayes, et al. and assigned to Microfab Technologies, Inc., we are taught that an ink-jet dispenser is used for the micro-dispensation of airborne materials into the inspired airstream or personal airspace of a subject. Preferably the airborne materials are presented in the form of a drug, fragrance or a substance comprising a volatile component. This patent does not disclose methods for mixing scents, actuation over the Internet, nor does this patent synchronously deliver both recorded images and scent.

U.S. Pat. No. 6,371,451 for a Scent Diffusion Apparatus and Method issued to Choi and assigned the Korea Institute of Science and Technology discloses an apparatus and method for consisting of an ink jet printer, cartridges for scents, and a database, the combination of which is used to mix and spray scents onto a subject. This patent does not disclose an apparatus or methods for electronic or Internet based actuation of the mixing of scents, nor does the patent disclose a method for synchronously delivering mixed scent and digital images to a recording apparatus, nor does this patent provide a means for spraying scent in targeted areas on recording subject.

3. Disadvantages of Prior Arts

In the above referenced prior art, various attempts have been made to spray scent, some with recorders, others with ink jet dispensers and a plurality of cartridges, and even one with use of a database so as to deliver precise and predictable mixing and spraying of scent, and one with proportional flow control valves to deliver precise and predictable mixing and spraying of scent. However, unlike the present invention which provides for the following capabilities, none of the above referenced prior arts disclose a method, means, or apparatus for these significant and distinguishing achievements of the present invention;

1) Remote Electronic Signaling and Actuation of Scent Mixing and Scent Spraying using the apparatus in conjunction with a single microprocessor or over a network of distributed computers, such as over the Internet,

2) Synchronous Delivery of Digitized Image recording commands and Digitized Scent Mixing and Scent Spraying commands using the apparatus in conjunction with a single microprocessor or over a network of distributed computers, such as over the Internet,

3) Spraying of Scent into Targeted Areas onto the addressable targeted areas, including by way of example, and not limited to, Pixels Inside Recorded Characters and recorded computer generated images, graphic art, line art, and text images in areas targeted for directed image and scent exposure to the user,

4) Data Management to provide search and data mining capabilities with a single personal computer or over a network of distributed computers, such as over the Internet, and

5) The delivery of a robust array of derivative scents, made possible by the construct of one or more multi-compartmentalized basic scent vessels.

As an original body of work previously non-disclosed, the present invention is both non-obvious, to someone of ordinary skill in the art, and useful in that it not only combines old elements in a new way to produce a new result and overcome pre-existing problems, such as increasing the basic utility of digital devices such as ink jet, laser and light emitting diode (LED) recorders to produce scent, but compelling advantages of the present invention over prior art include that it provides a useful method for consumers to easily, rapidly, cost efficiently, and at any hour of the day deploy the advanced power of distributed network computing to synchronously, in real time, associate Internet based images of products with digitized scents, thereby empowering consumer choice for users shopping over the Internet to add scent as a product evaluation criteria. In addition, other compelling advantages that the present invention has over prior art is that it provides product manufacturers and marketers with the ability to easily, rapidly, cost efficiently, and at any hour of the day add or change a robust array of scents to their product branding platform, increase brand awareness through imprinting the olfactory senses of end users by targeting the location and proximity of scent with recorded brand images, and add related data management capabilities to better target the needs of consumers, using the Internet in a new way to distinguish their products and to appeal to a greater audience. The present invention is also economical to produce.

### BRIEF SUMMARY OF THE INVENTION

1) It is an objective of the present invention to provide Remote Electronic Signaling and Actuation of Scent Mixing and Scent Spraying using the apparatus in conjunction with a single microprocessor or over a network of distributed computers, such as over the Internet. Moreover, it is an objective to provide a computer implemented scent formulation method and scent diffusion system,

2) It is an objective of the present invention to provide Synchronous Delivery of Digitized Image recording commands and Digitized Scent Mixing and Scent Spraying commands using the apparatus in conjunction with a single microprocessor equipped with an applications software module or over a network of distributed computers equipped with applications software modules, such as over the Internet. Moreover, it is an objective to provide a scent formulating and scent diffusing apparatus that interacts with a database for synchronously mixing precise amounts of one or more scents, spraying one or more scents and recording one or more digital images upon electronic signal demand,

3) It is an objective of the present invention to provide Spraying of One or More Scents onto Targeted Areas, including by way of example, and not limited to, the addressable Pixels, as identified by a database, Inside recorded Characters and recorded computer generated images, graphic art, line art, and text images, in areas targeted for directed image and scent exposure to the user,

4) It is an objective of the present invention to provide Data Management to provide search and data mining capabilities with a single personal computer or over a network of distributed computers, such as over the Internet. Moreover, it is an objective to provide the methods, systems, apparatus, computer programs and compositions to create and access a hierarchical database optimally suited for use in scent mixing and scent archival. It is an objective to also provide the ability to mine data from a database containing a plurality of chemical fingerprints.

5) It is an objective of the present invention to provide the delivery of a Robust Array of Derivative Scents, made possible by the construct of one or more multi-compartmentalized basic scent vessels.

In order to realize the above objectives, the present invention involves a scent formulating and scent diffusing apparatus including, but not limited to, a head assembly spraying unit which slides on a rail mounted inside the recorder, and multiple scent vessels attached to one side of the head assembly containing various scents. A user selected digital image and image recording command appear on a computer screen, and corresponding digital scent mixing and spraying commands are synchronously, along with the user initiated image recording command, sent, by means of an application software module, to the computer controller, where scent mixing commands interact with a database to gather precise volume mix data and spray location coordinates and whereby, the computer controller commands the recorder's head assembly spray unit to extract scented oils in exact amounts and mix such scented oils thereby producing derivative scents, for subsequent spraying onto recording subject while the head assembly spraying unit rotating nozzle is moved by a belt during the recording process.

### BRIEF DESCRIPTION OF THE FIGURES

A more complete understanding of the objects and advantages of the present invention becomes attainable by reference to the following drawings and the detailed description.

FIG. 1 is a schematic diagram of the scent formulating and scent diffusing apparatus according to a preferred embodiment of the present invention.

FIG. 2 is a block diagram of the scent formulating and scent diffusing apparatus according to a preferred embodiment of the present invention.

FIG. 3 shows the multi-compartmentalized scent vessel A and scent formulation of FIG. 1 in greater detail.

FIG. 3a shows the third preferred embodiment of a dual function ink-scented oil sub-compartmentalized vessel.

FIG. 4 shows the head assembly spraying unit B, rail C, belt D, ink head E and scent diffusion of FIG. 1 in greater detail.

FIG. 5 shows the head assembly spraying scent mixing chamber, piezoelectric transducer, air pump, and air flow of an air-gas mixture of FIG. 4 in greater detail

FIG. 6 is a flowchart of a distributed computing environment for the scent formulating and scent diffusing apparatus according to a preferred embodiment of the present invention.

FIG. 7 is a flowchart of the control sequence for image recording instruction formation and delivery, and scent formulating and scent diffusing by the apparatus according to a preferred embodiment of the present invention.

FIG. 8 is a Data Management Flowchart for the scent formulating and scent diffusing apparatus according to a preferred embodiment of the present invention.

### DETAILED DESCRIPTION OF PREFERRED EMBODIMENTS OF THE INVENTION

In order to realize the above objectives, the present invention involves a scent formulating and scent diffusing apparatus for synchronously mixing one or more scents, spraying one or more scents, and recording a digital image upon electronic signal demand, with said signaling mechanism either located directly within a common commercially available microprocessor, such as a personal computer, or over a network of distributed computers such as the world wide web, commonly referred to as the Internet, comprising; a controller, commonly found in commercially available microprocessors also known as personal computers, separate from the apparatus, generating a signal on demand from the controller, an input of the apparatus accepting the signal.

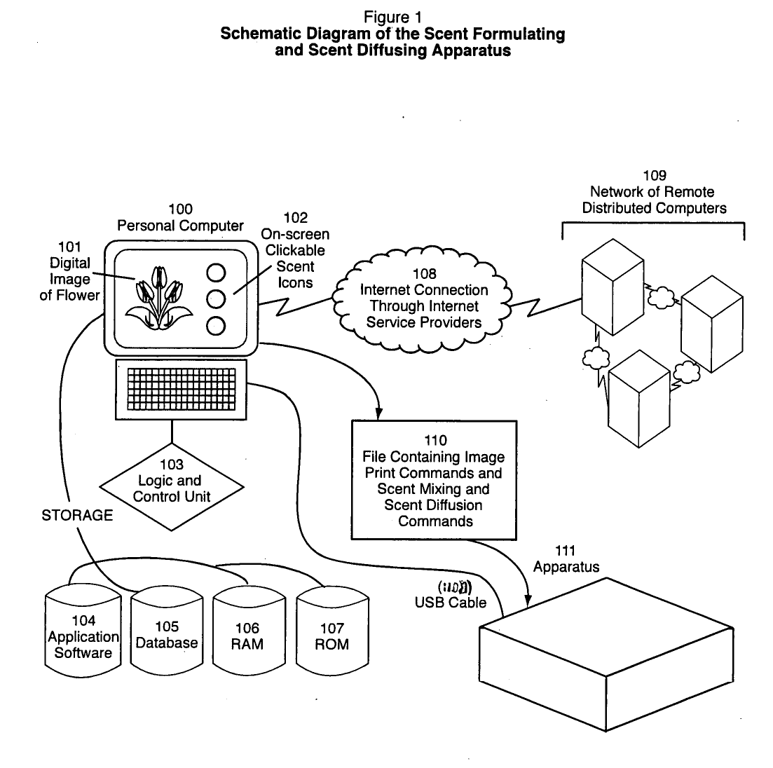

FIG. 1 is a schematic diagram of the scent formulating and scent diffusing recording apparatus 111 according to a preferred embodiment of the present invention where, as a user is looking at and desiring to actuate the synchronous 110 recording of a specific digital image 101 appearing on the screen of his microprocessor 100, the formulating of a specific scent 102 associated with said digital image, and the diffusing of that specific scent associated with said digital image appearing on the screen of his microprocessor 100, with said microprocessor interacting with a remotely located computer 109 or a network of distributed remotely located computers 109 such as the world wide web, commonly referred to as the Internet 108, with said microprocessor and remotely located computer or computers being outfitted with application software module 104 and its database 105, user's interaction with microprocessor resident application software module 104 and its database 105 causes subsequent simultaneous interaction with the logic and control unit 103 of the remotely located computer(s) 109 and with the logic and control unit 103 of the user's said microprocessor 100, which in turn, causes an electronic signal to synchronously actuate both an image recording command and scent formulating and scent diffusion commands, associated with the image, to be delivered to and accepted by the controller interface and logic and control unit located in the scent formulating and scent diffusing recording apparatus 111. It is understood that each logic and control unit consists of a series of separate functional elements including central processing unit (CPU), temporary random access memory (RAM) 106 also referred to as execution memory, and read only memory (ROM) 107 also referred to as program memory. It is further understood to those skilled in the art, that digital images 101 include computer generated images, graphic art, line art, and text images and the like.

In a second embodiment of the present invention according to FIG. 1, where, as a user is looking at and desiring to actuate the synchronous recording of a specific digital image 101 appearing on the screen of his microprocessor 100, the formulating of a specific scent associated with said digital image 101, and the diffusing of a specific scent 102 associated with said digital image 101 appearing on the screen of his microprocessor 100, with said microprocessor not interacting with a remotely located or network of distributed remotely located computers, and with said microprocessor being outfitted with application software module 104 and its database 105, user's interaction with application software module 104 and its database 105 causes subsequent interaction with the logic and control unit 103 of the user's said microprocessor 100 causes an electronic signal to synchronously actuate both an image recording command and scent formulating and scent diffusion commands, associated with the image, to be delivered and accepted by the controller interface and logic and control unit located in the scent formulating and scent diffusing recording apparatus.

In FIG. 1, a graphics card and corresponding on-screen icon image 102, commonly found in commercially available microprocessors 100, when actuated, synchronously transfers both digital image recording commands and digital scent diffusion commands to a computer software module 104 which, in turn, formats, synchronizes and transfers digital image recording commands and digital scent mixing and digital scent diffusion commands 110 to the recorder apparatus 111 upon demand as a function of the signal.

As shown in FIG. 1, in a preferred embodiment of the present invention, there is provided an apparatus resident CPU which receives a digital image 101 from the microprocessor 100. In the preferred embodiment, the means to actuate the synchronous transfer of both digital image recording commands and scent formulating and scent diffusion commands 110, associated with said digital image 101, from the microprocessor CPU 103 and the application software module 104 to the apparatus controller interface and logic and control unit located in the scent formulating and scent diffusing recording apparatus 111, is achieved by virtue of user input into application software module 104. Upon user input, software module 104 creates in the microprocessor 100 CPU 103, saves in the microprocessor 100 random access memory (RAM) memory 106, and subsequently transfers from the microprocessor 100 RAM 106 to the apparatus' 111 RAM memory, an image file 110 containing said digital image 101, said digital image 101 recording commands, said image specific scent formulation commands, and said image specific scent diffusion commands, with application software module 104 accessing look-up tables in its database 105 to identify, retrieve and save in said image file 110, previously stored data representative of the derivative scent formula, associated with the digital image 101, and the identity and amount of such basic constituent scented oils to be mixed to achieve the derivative scent, and data with the location coordinates of where the digital image 101 is to be recorded and location coordinates of where the scent is to be diffused relative to where the digital image 101 is to be recorded. Among other data, the digital image recording commands include number of digital pixels which represents a continuous tone colored image, made form a spectrum of derivative colors using the base colors of Magenta, Cyan, Yellow, and Black.

A second preferred embodiment (not shown) in the present invention as a means to achieve synchronous delivery of digital image recording command and scent formulation and scent diffusion commands is the use of USB for data interconnection between the microprocessor and the apparatus. In the book “PC 97 Hardware Design Guide”, Microsoft deprecated support for the RS-232 compatible serial port of the original International Business Machines Corporation (IBM )PC design. Today, RS-232 is gradually being superseded in personal computers by USB. Compared with RS-232, USB is faster, has lower voltage levels, has connectors that are simpler to connect and use. USB has software support in popular operating systems. USB is designed to make it easy for device drivers to communicate with hardware, and there is no direct analog to the terminal programs used to let users communicate directly with serial ports. However, USB is a more complex standard than RS-232, requiring more software to support the protocol used.

A third preferred embodiment (not shown) to provide synchronous delivery of image recording commands and scent formulation and scent diffusion commands from the microprocessor to the scent formulating and scent diffusing recorder apparatus is to use RS-232 serial binary data interconnection whereby the microprocessor acts as a piece of data terminal equipment (DTE) and the apparatus serves as a piece of data communication equipment (DCE). RS-232 is commonly used in computer serial ports. A similar ITU-T standard is V.24. The EIA standard RS-232-C as of 1969 defines; Electrical signal characteristics such as voltage levels, signaling rate, timing and slew-rate of signals, voltage withstand level, short-circuit behavior, maximum stray capacitance and cable length; Interface mechanical characteristics, pluggable connectors and pin identification; Functions of each circuit in the interface connector; and Standard subsets of interface circuits for selected applications. The standard does not define such elements as character encoding (for example, ASCII, Baudot or EBCDIC), or the framing of characters in the data stream (bits per character, start/stop bits, parity). The standard does not define bit rates for transmission, although the standard says it is intended for bit rates less than 20,000 bits per second. Many modern devices can exceed this speed (38,400 and 57,600 bit/s being common, and 115,200 and 230,400 bit/s making occasional appearances) while still using RS-232 compatible signal levels.

Many personal computers intended for office use ship with “legacy-free” motherboards without any RS-232 serial ports. However, RS-232 is still quite common in point-of-sale (cash drawers, barcode and magnetic stripe readers), amateur electronics and industrial measurement and remote-control devices, so computers made for such applications are still equipped with RS-232 ports. As an alternative, USB docking ports are available which can provide connectors for a keyboard, mouse, one or more serial ports, and one or more parallel ports. Corresponding device drivers are required for each USB-connected device to allow programs to access these USB-connected devices as if they were the original directly-connected peripherals.

It is also possible to connect RS-232 devices via ethernet and WLAN device drivers that act as network servers. Some manufacturers even have virtual serial port drivers available.

In RS-232, data is sent as a time-series of bits. Both synchronous and asynchronous transmissions are supported by the standard. Each circuit only operates in one direction, that is, signaling from a DTE to the attached DCE or the reverse. Since transmit data and receive data are separate circuits, the interface can operate in a full duplex manner, supporting concurrent data flow in both directions. The standard does not define character framing within the data stream, or character encoding.

The most common arrangement, nearly universal in personal computers, is an asynchronous link sending seven or eight bits. When used in this way, the bit order consists of a start bit, seven or eight data bits sent least significant bit first, an optional parity bit, and a stop bit. The steady state of the line is called the Marking state. The start of a new character is signaled by a change to the Space state. The digital ones and zeros are then transmitted serially onto the line by switching between the Mark and Space state ending with a final stop bit which is a transition back to the Marking state. Mechanical telerprinter often required extra time to ready themselves for the next character, so many systems could be arranged to send two stop bits.

Historically, teleprinter and related devices used ASCII in the US, or various character codes elsewhere. Some very old telerprinter and Telex systems use Baudot code.

Voltage Levels

The RS-232 standard defines the voltage levels that correspond to logical one and logical zero levels. Signals are plus or minus 3 to 15 volts. The range near zero volts is not a valid RS-232 level; logic one is defined as a negative voltage, the signal condition is called marking, and has the functional significance of OFF. Logic zero is positive, the signal condition is spacing, and has the function ON. The standard specifies a maximum open-circuit voltage of 25 volts; signal levels of ±5 V, ±10 V, ±12 V, and ±15 V are all commonly seen depending on the power supplies available within a device. Circuits driving an RS-232-compatible interface must be able to withstand indefinite short circuit to ground or to any voltage level up to 25 volts. The slew rate, or how fast the signal changes between levels, is also controlled.

Connectors

RS-232 devices may be classified as Data Terminal Equipment (DTE) or Data Communications Equipment (DCE); this defines which wires will be sending and receiving each signal. The standard recommended but did not make mandatory the common D-subminiature 25 pin connector. In general, terminals have male connectors with DTE pin functions, and modems have female connectors with DCE pin functions. Other devices may have any combination of connector gender and pin definitions.

Presence of a 25 pin D-sub connector does not necessarily indicate an RS-232C compliant interface. For example, on the original IBM PC, a male D-sub was an RS-232C DTE port (with a non-standard current loop interface on reserved pins), but the female D-sub connector was used for a parallel Centronics recorder port. Some personal computers put non-standard voltages or signals on their serial ports.

The standard specifies 20 different signal connections. Since most devices use only a few signals, smaller connectors can be used. For example, the 9 pin DE-9 connector was used by most IBM-compatible PCs since the IBM PC AT. More recently, 8 pin RJ-45 connectors have become common, although pin assignments vary widely. Standard EIA/TIA 561 specifies a pin assignment, but the “Yost Serial Device Wiring Standard” invented by Dave Yost is common on Unix computers, and many devices don't follow either of these. 10 pin RJ-50 connectors can be found on some devices as well.

The following table lists the commonly used RS-232 signals and common pin assignments (see also RS-485 for different standard with the same connectors):

| Signal | DB-25 | DE-9 | EIA/TIA 561 | Yost | RJ-50 |
|---|---|---|---|---|---|
| Common Ground | 7 | 5 | 4 | 4,5 | 6 |
| Transmitted Data (TD) | 2 | 3 | 6 | 3 | 8 |
| Received Data (RD) | 3 | 2 | 5 | 6 | 9 |
| Data Terminal Ready | 20 | 4 | 3 | 2 | 7 |
| (DTR) | | | | | |
| Data Set Ready (DSR) | 6 | 6 | 1 | 7 | 5 |
| Request To Send (RTS) | 4 | 7 | 8 | 1 | 4 |
| Clear To Send (CTS) | 5 | 8 | 7 | 8 | 3 |
| Carrier Detect (DCD) | 8 | 1 | 2 | 7 | 10 |
| Ring Indicator (RI) | 22 | 9 | 1 | — | 2 |

The signals are labeled from the standpoint of the DTE device; TD, DTR, and RTS are generated by the DTE and RD, DSR, CTS, DCD, and RI are generated by the DCE. The ground signal is a common return for the other connections; it appears on two pins in the Yost standard but is the same signal. Connection of pin 1 (protective ground) and pin 7 (signal reference ground) is a common practice but not recommended. Use of a common ground is one weakness of RS-232. If the two pieces of equipment are far enough apart or on separate power systems, the ground will degrade between them and communications will fail; this is a difficult condition to trace.

Note that EIA/TIA 561 combines DSR and RI, and the Yost standard combines DSR and DCD.

Cables

Since the standard definitions are not always correctly applied, it is often necessary to consult documentation, test connections with a breakout box, or use trial and error to find a cable that works when interconnecting two devices. Connecting a fully-standard-compliant DCE device and DTE device would use a cable that connects identical pin numbers in each connector (a so-called “straight cable”). “Gender changers” are available to solve gender mismatches between cables and connectors. Connecting devices with different types of connectors requires a cable that connects the corresponding pins according to the table above. Cables with 9 pins on one end and 25 on the other are common, and manufacturers of equipment with RJ-45 connectors usually provide a cable with either a DB-25 or DE-9 connector (or sometimes interchangeable connectors so they can work with multiple devices).

Connecting two DTE devices together requires a null modem that acts as a DCE between the devices by swapping the corresponding signals (TD-RD, DTR-DSR, and RTS-CTS). This can be done with a separate device and two cables, or using a cable wired to do this. One feature of the Yost standard is that a null modem cable is a “rollover” cable that just reverses pins 1 through 8 on one end to 8 through 1 on the other end.

For configuring and diagnosing problems with RS-232 cables, a “breakout box” may be used. This device normally has a female and male RS-232 connector and is meant to attach in-line; it then has lights for each pin and provisions for interconnecting pins in different configurations.

RS-232 cables may be built with connectors commonly available at electronics stores. The cables may be between 3 and 25 pins; typically 4 or 6 wires are used. Flat RJ (phone-style) cables may be used with special RJ-RS232 connectors, which are the easiest to configure.

The reason that a minimal two-way interface can be created with only 3 wires is that all the RS-232 signals share a common ground return. The use of unbalance circuits makes RS-232 susceptible to problems due to ground potential shifts between the two devices. RS-232 also has relatively poor control of signal rise and fall times, leading to potential crosstalk problems. RS-232 was recommended for short connections (15 meters or less). RS-232 interface cables are not usually constructed with twisted pair because of the unbalanced circuits.

While the control lines of the RS 232 interface were originally intended for call setup and takedown, other “handshakes” may be required by one or the other device. These are used for flow control, for example, to prevent loss of data sent to a serial recorder. For example, pin 20 is commonly used to indicate “device ready”. Pins may also be “jumpered” or routed back within the connector. For example a pin saying “are you ready?” from device A might be wired to the pin saying “I'm ready” on device A if device B did not transmit such a signal. Common handshake pins are 20, 8, 4, and 6.

Signal Rate Selection

The DTE or DCE can specify use of a “high” or “low” signaling rate. The rates as well as which device will select the rate must be configured in both the DTE and DCE. The prearranged device selects the high rate by setting pin 23 to ON.

Loopback Testing

Many DCE devices have a loopback capability used for testing. When enabled, signals are echoed back to the sender rather than being sent on to the receiver. If supported, the DTE can signal the local DCE (the one it is connected to) to enter loopback mode by setting pin 18 to ON, or the remote DCE (the one the local DCE is connected to) to enter loopback mode by setting pin 21 to ON. The latter tests the communications link as well as both DCE's. When the DCE is in test mode it signals the DTE by setting pin 25 to ON.

A commonly used version of loopback testing doesn't involve any special capability of either end. A hardware loopback is simply a wire connecting complimentary pins together in the same connector.

Loopback testing is often performed with a specialized DTE called a Bit Error Rate Tester (BERT).

Timing Signals

Some synchronous devices provide a clock signal to synchronize data transmission. The timing signals are provided by the DCE on pins 15 and 17. Pin 15 is the transmitter clock; the DTE puts the next bit on the data line (pin 2) when this clock transitions from OFF to ON (so it is stable during the ON to OFF transition when the DCE registers the bit). Pin 17 is the receiver clock; the DTE reads the next bit from the data line (pin 3) when this clock transitions from ON to OFF.

Alternatively, the DTE can provide a clock signal on pin 24 for both transmitted and received data. Again, data is changed when the clock transitions from OFF to ON and read during the ON to OFF transition

In electronics and synchronous digital circuits, such as most computers, a clock signal is a signal used to coordinate the actions of two or more circuits. A clock signal oscillates between a high and a low state, normally with a 50% duty cycle, and is usually a square wave. The circuits using the clock signal for synchronization may become active at either the rising or falling edge, or both (for example DDR SDRAM), of the clock signal.

Some sensitive mixed-signal circuits, such as precision analog-to-digital converters, use sine waves rather than square waves as their clock signals, because square waves contain high-frequency harmonics that can interfere with the analog circuitry and cause noise. Such sine wave clocks are often differential signals, because this type of signal has twice the slew rate, and therefore half the timing uncertainty, of a single-ended signal with the same voltage range.

Most integrated circuits of sufficient complexity require a clock signal in order to synchronize different parts of the chip and to account for propagation delays. As chips get more complex, the problem of supplying accurate and synchronized clocks to all the circuits becomes more and more difficult. The preeminent example of such complex chips is the microprocessor, the central part of modern computers.

A clock signal might also be gated, i.e. combined with a controlling signal, in order to enable or disable the clock signal for a certain part of a circuit. This technique is often used to save power by shutting down parts of a digital circuit currently not being used.

In some early microprocessors such as the National Semiconductor IMP-16 family, a multi-phase clock was used. In the case of the IMP-16, the clock actually had four phases, each 90 degrees apart, in order to synchronize the operations of the processor core and its peripherals. Most modern microprocessors and microcontrollers use a single phase clock.

Some modern computers, notably PCs, use the concept of a “clock multiplier” which takes a lower frequency external clock and multiplies it to the appropriate clock rate of the processor chip. This allows motherboard manufacturers to build one board that can accommodate various speeds of processor simply by altering the clock multiple. The speed of a clock signal in a computer is called the clock rate or clock frequency.

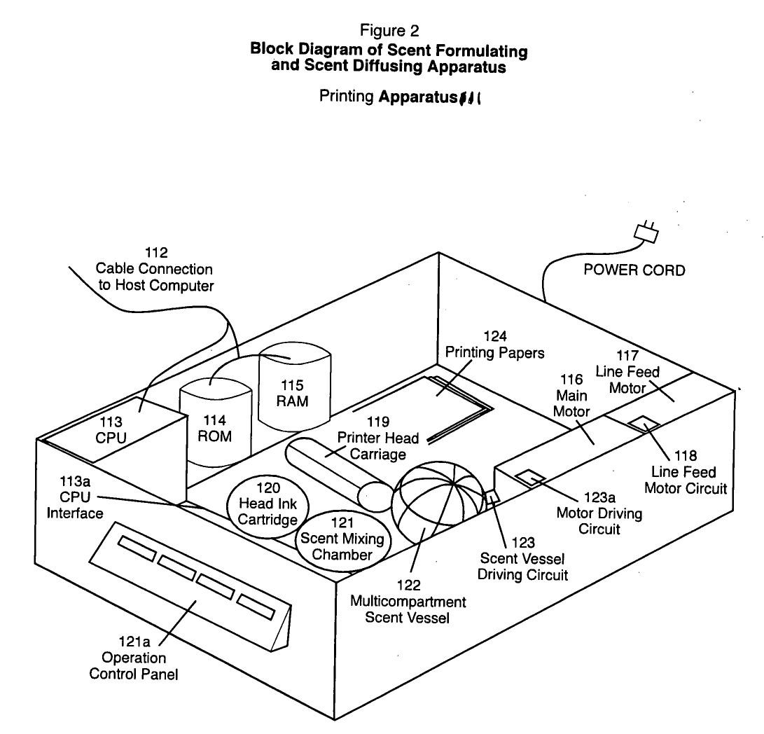

FIG. 2 is a block diagram of the scent formulating and scent diffusing recording apparatus 111 according to a preferred embodiment of the present invention where, a scent vessel driving circuit 123 and a scent vessel cartridge 122 are added to a conventional ink jet recorder. A CPU 113 controls the overall operation of the recorder according to a program stored in a read only memory (ROM) 114. ROM 114 stores the operating program for CPU 113 and initial data. A random access memory (RAM) 115 temporarily stores data produced through the operation of CPU 113. An interface 113a links CPU 113 to components of the recorder. A computer interface 112 provides a parallel type interface for receiving data from a host computer 100 (FIG. 1). An operation panel 121a includes a set of keys by which control commands are fed into the recorder, and a display that displays status information during the recording operation. Motor driving circuit 123a drives a main motor 116 under the control of CPU 113. Motor driving circuit 123a drives a carriage convey motor (hereinafter, referred to as CR motor) 119 under the control of CPU 113. Carriage convey motor 119, driven by motor driving circuit 123a, moves a head cartridge 120 in a horizontal direction. A motor driving circuit 123a is controlled by CPU 113 to drive a line feed (LF) motor 117. Line feed motor 117, responding to motor driving circuit 123a, conveys recording papers 124. A head driving circuit drives a head cartridge 120 under the control of CPU 113.

Head cartridge 120 responds to signals from head driving circuit to spray ink particles onto the recording papers 124. Scent vessel driving circuit 123 controls scent vessel cartridge 122, under the direction of CPU 113. Scent vessel cartridge 122, driven by scent vessel driving circuit 123, transfers one or more constituent scented oils to the mixing chamber.

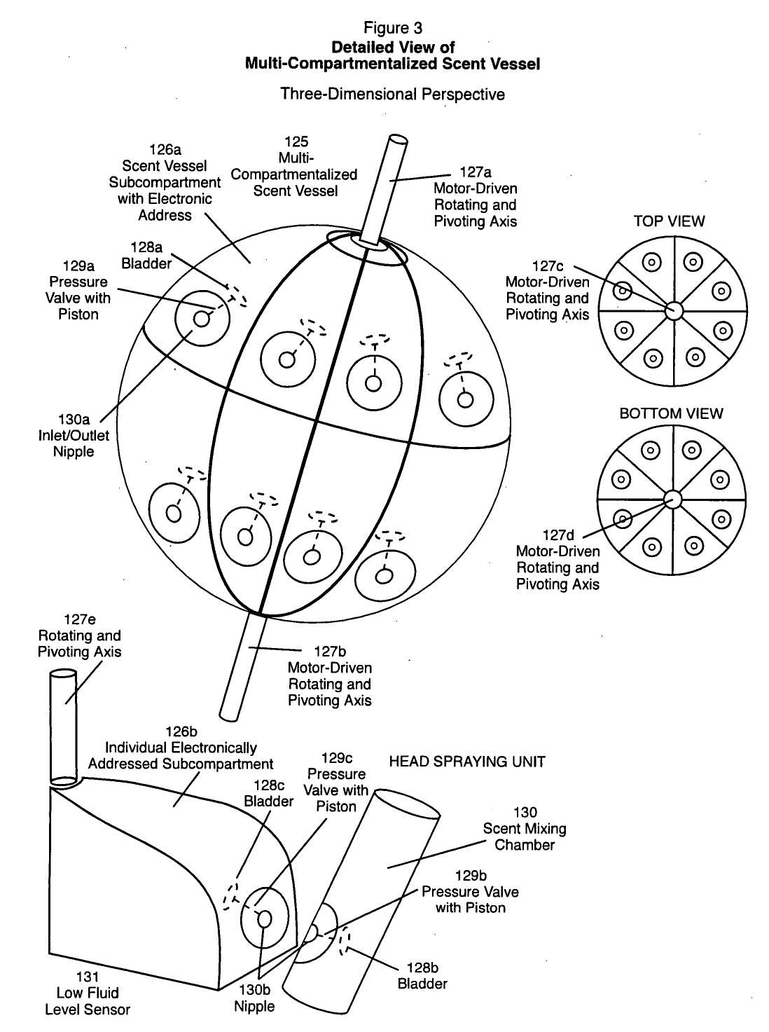

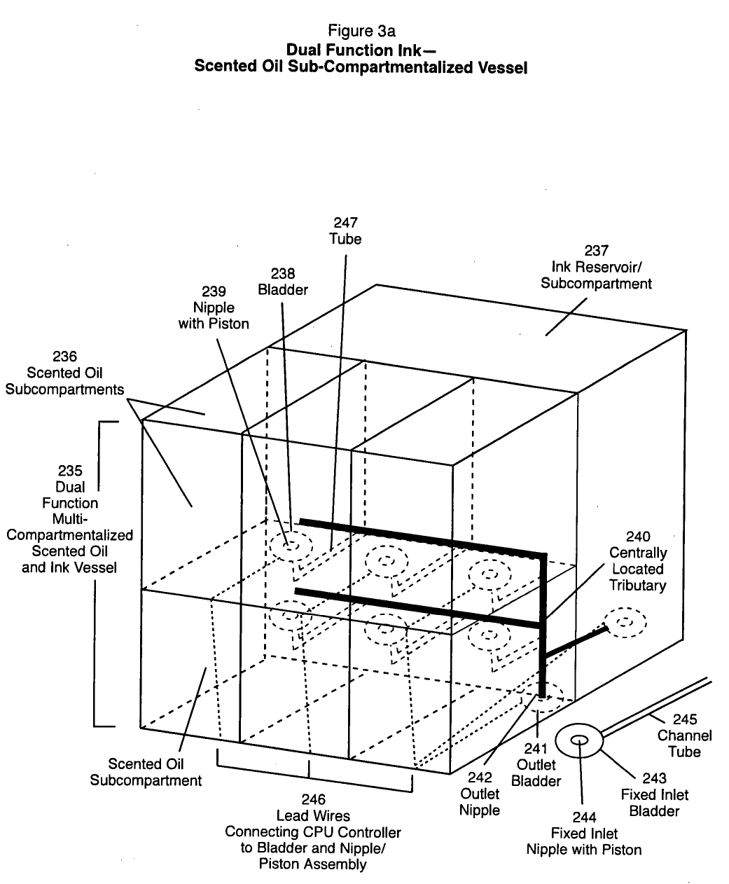

FIG. 3 is a detailed view of the multi-compartmentalized scent vessel 125 and scent formulation. The scent formulating and scent diffusing recorder apparatus has one or more scent vessel assemblies 125, each capable of being sub-divided with compartmentalized walls 126a, wherein stored inside of said one or more scent vessel assemblies 125 are a plurality of individual scented oils, and where said one or more scent vessel assemblies 125, under control of the microprocessor 100 CPU 103, application software module 104, and apparatus control interface 113a, transfers precise predetermined amounts of one or more constituent scented oils to the mixing chamber 121, 130.

As shown in FIG. 3, the preferred embodiment of the present invention provides one or more scent vessels 125, illustrated as one or more moving, interchangeable, spherical shaped, multi-compartmentalized scent vessels 125, each housed on a motor driven rotating and pivoting axis 127a with 2 points of contact with the spherical shaped scent vessel 125, has a provision for an electronic address to identify sub-divided scent vessel assembly locations 126a where basic scented fragrant oils are stored. The use of spherical shaped multi-compartmentalized scent vessels 125 was partially inspired by the 1961 invention of the International Business Machines Corporation (IBM) Seletcric I “golf-ball” typewriter recording head, designed by Elliott Noyes (see website of the Industrial DesignersSociety-America at www.idsa.org/webmodules/articles/anmviewer.asp?a=264). There is also provision for an electronic benchmark sensor 131 located inside each sub-divided scent vessel assembly 126b for purposes of indicating remaining levels of scented fragrant fluid inside the sub-divided scent vessel assemblies 126b. On each sub-compartmentalized unit 126b of each sphere shaped scent vessel 125, there is a bladder 128c, nipple 130b, pressure valve with piston 129b, each connected to each sub-divided scent vessel assembly 126a for purposes of releasing and transporting precise amounts of scented fragrant oil from each sub-divided scent vessel assembly 126b to the scent mixing chamber 130 within the head assembly spraying unit.

Upon receipt of an electronic signal from the microprocessor 100 CPU 103, application software module 104, and apparatus controller interface 110a, in a serial fashion, the spherical scent vessel 125 is rapidly rotated on its pivoting axis 127a by means of a motor 116, with said rotation stopping when a scent vessel-sub-compartment 126b bearing an address that matches the identity of basic constituent scented oils, as predetermined by the application software module 104 database 105, has its nipple 130b positioned for direct connection to the head spray assembly unit's 130 scent chamber 130 intake valve 129b on the spraying unit assembly, the spraying unit assembly 130 of which, under control of the apparatus' CPU 113, slides into position and then remains fixed during the impregnation process, whereby a precise amount of each database 105 selected scented constituent oil from each sub-divided scent vessel assembly 126b is released and transported into the adjacent scent mixing chamber 130.

To aid in the efficient flow of basic scented oil, and to reduce oils spillage or trapping during transport, the rapid spinning motion of the spherical scent vessel 125 creates centrifugal force inside of scent vessel sub-compartments 126a, whereby resident basic oils contained inside the scent vessel sub-compartments 126a are drawn to the outmost wall of said sub-compartmentalized scent vessel 126a, with said centrifugal force creating adequate pressure to help oils flow in the direction of the pressure valve 129a, which is located on the lower outermost wall of each scent vessel sub-compartment 126a, and which is positioned by the apparatus in direct contact with the scent chamber 130 intake valve 129b for purposes of transporting said oils to scent mixing chamber 130 for mixing. Moreover, by positioning the pressure valve 129a on the lower outermost wall of each scent vessel sub-compartment 126a, desired scented oil movement toward the pressure valve 129a is aided by centrifugal force that push the scent oils contained therein to the outside walls as noted, and desired scented oil movement toward the pressure valve 129a is aided by gravitational forces that pull scented oils downward toward the area inside the scent vessel sub-compartment 126a where the pressure valve 129a is located. In order to assure the proper constituent mix of basic scent oils needed to produce a derivative formulated scented oil, the spherical scent vessel 125 rotating process is repeated in successive iterations with each addressable scent vessel sub-compartment 126a as necessary. The pressure valve 129a, nipple 130a, and bladder assembly 128a serves as a means of both inletting scent oils to initially impregnate the scent vessel 125, and subsequently as a means of releasing scented oils for transport out of the scent vessel 125 to the scent mixing chamber 130 as discussed in the aforementioned process above. The present invention of the spherical shaped multi-compartrnentalized scent vessels 125, each housed on a motor driven rotating and pivoting axis 127a with 2 points of contact with the spherical scent vessel 125 makes practical and economical the production of a robust numeric array of derivative scented oils. In example, as illustrated in FIG. 3, with 16 sub-compartments in the spherical scent vessel shown, each sub-compartment filled with a different basic scented oil, a matrix of up to 256 oil type combinations are available for mixing by the apparatus to produce thousands of derivative scented oil blends based on varying concentrations of the 256 oil type combinations.

In a second preferred embodiment (not shown)of the present invention, the number of derivative scented oil blends increases geometrically when either further subdividing the spherical scent vessel into additional sub-compartments, or when introducing a second, third, fourth, etc . . . , spherical scent vessel of similar configuration, while maintaining one spraying unit assembly that moves into position, under control of the apparatus' CPU to become impregnated by each of the applicable scent vessel sub-compartments, thus increasing a wide spectrum of possible scents created by the apparatus.

As shown in FIG. 3a, in order to utilize and retrofit the embedded base of pre-existing diffusion devices in the marketplace that already contain an ink jet cartridge, such as printers, the third preferred embodiment of the present invention provides one or more dual purpose ink-scented oil vessels 235, illustrated as one or more fixed, interchangeable, replaceable, rectangular shaped, multi-compartmentalized ink and scent vessels 235, with each sub-compartment having its own bladder 238, nipple-piston assembly 239 and tube 247, located at the lower most inner wall of each hsub-compartment 236 to utilize gravity to aid in liquid flows, and with said tube 247 dedicated for transport of liquid to a centrally located, commonly connected tributary 240. Each multi-compartmentalized ink and scent vessel 235 has one type of liquid ink housed in a sub-compartment 237 separate and apart from the several other sub-compartments 236 that contain scented oil liquids. Each multi-compartmentalized ink and scent vessel 235 has its centrally located tributary 240 positioned upon a fixed outlet bladder 241, nipple and piston assembly 242 internal to each multi-compartmentalized ink and scent vessel 235. The multi-compartmentalized ink and scent vessel 235 has its outlet nipple 242 connected to a fixed receiving nipple assembly 244 that in turn is connected to a channel tube 245 for transporting, in a serial fashion, combinations of scented oils, into the head assembly spraying unit 130 for subsequent diffusion, and then ink is transported into the head assembly spraying unit 130 for subsequent diffusion. Each multi-compartmentalized ink and scent vessel 235 has a provision for an electronic address to identify sub-divided ink and scent vessel assembly locations 126a where inks and basic scented fragrant oils are stored. Ink and scented oils are not co-located in each vessel; rather, inks are located separately in electronically sub-divided vessels, and scented oils are located in separately in electronically sub-divided vessels. There is also provision for an electronic benchmark sensor 131 located inside each sub-divided scent vessel assembly 126b for purposes of indicating remaining levels of ink and scented fragrant fluid inside each sub-divided scent vessel assemblies 126b.

Leading from each sub-divided vessel is a tube 247, placed internal to the perimeter walls of the overall ink-scented oils vessel 235, whereby said tube 247 serves as a means of transport to move inks and scented oils, under control of the apparatus CPU 113, releases and transports, in a serial fashion, precise amounts of ink from each sub-divided vessel assembly 126b to the mixing chamber 130 within the head assembly spraying unit. After the ink spraying cycle is complete, under control of the apparatus CPU 113, the apparatus CPU 113 signals a subsequent release and transport of precise amounts of scented fragrant oil from each sub-divided scent vessel assembly 126b to the scent mixing chamber 130 within the head assembly spraying unit. On the bottom side of each of each rectangular shaped ink-scented oil vessel 125, there is a bladder 128c, nipple 130b, pressure valve with piston 129b, each connected to each ink-scented oil vessel assembly 126a for purposes of releasing and transporting precise amounts of liquid from each vessel.

Upon receipt of an electronic signal from the microprocessor 100 CPU 103, application software module 104, and apparatus controller interface 110a, in a serial fashion, the vessel-sub-compartment 126b bearing an address that matches the identity of either an ink or a basic constituent scented oil, as predetermined by the application software module 104 database 105, has its transport tube valve opened, thereby releasing the flow of liquid to the vessel nipple 130b positioned for direct connection to the apparatus' intake valve 129b which, under control of the apparatus' CPU 113, remains in a fixed open position during the impregnation process, whereby a precise amount of each database 105 selected liquid, either scented oil or ink, from each sub-divided scent vessel assembly 126b is released and transported into the adjacent apparatus nipple for subsequent transport to the head spraying mechanism. At the end of each spraying operation, under control of the apparatus CPU 113, the head assembly spraying unit is wiped clean by an armature outfitted with a cotton swap, or similarly absorbent material, treated with a solvent to eliminate residual inks and residual scented oils from the spraying head.

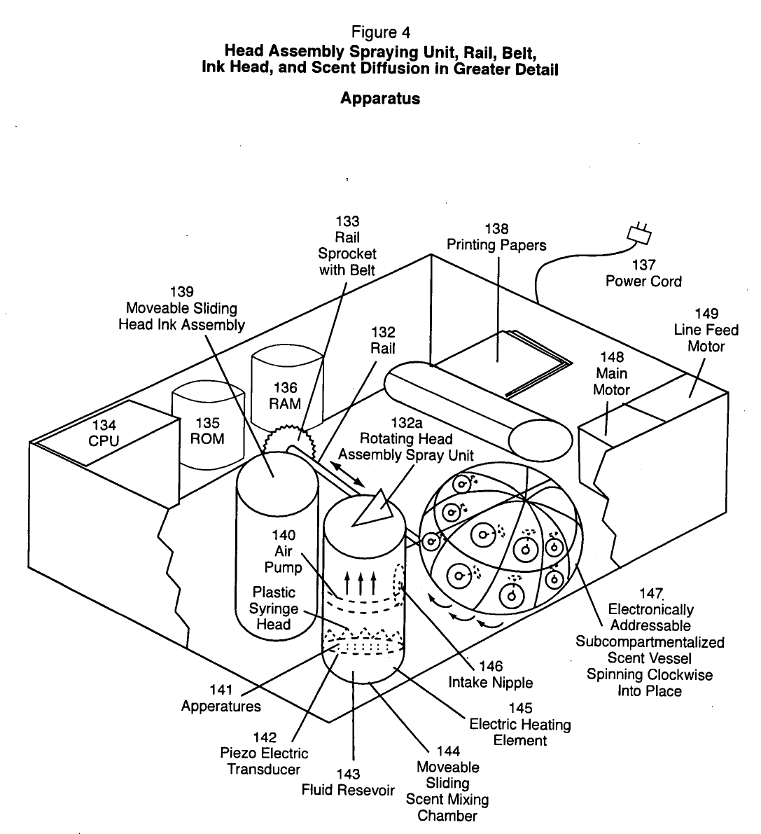

FIG. 4 shows a view of greater detail of the head assembly spraying unit 132a, rail 132, belt 133, ink head 139 and scent diffusion. FIG. 4 shows that the scent formulating and scent diffusing recorder apparatus has a belt 133; a rail 132 within the recorder to be moved by said belt 133; a head assembly spraying unit 132a sliding on said rail 132 within the recorder to spray scent on sheets of recording papers 138; and one or more scent vessel assemblies 147, each capable of being sub-divided with compartmentalized walls, located in the recorder, spaced apart from the head assembly spraying unit 132a and positioned to selectively spray scent on the recording papers 138 in a targeted region to be recorded on.

In addition, FIG. 4 shows that the scent formulating and scent diffusing recorder apparatus has a belt 133; a rail 132 within the recorder to be moved by said belt; an ink head assembly 139 sliding on said rail 132 within the recorder to recording ink on sheets of recording papers 138; and one or more scent vessel assemblies 147 located in the recorder, spaced apart from the head assembly spraying unit 139 and positioned to selectively spray scent on the recording papers 138 in a targeted region to be recorded on.

FIG. 4 also shows the scent formulating and scent diffusing recorder apparatus has a head assembly spraying unit 132a, wherein said spraying unit 132a is mounted in the recorder so as to selectively spray scent on pixels inside characters recorded on said recording papers on which said ink is recorded. The head assembly spraying unit 132a also receives and mixes precise predetermined amounts of one or more scented fragrant oils to produce a derivative scent, and, subsequently, diffusing said scent, with said head assembly spraying unit 132a, sliding, upon demand as a function of the signal, on said rail 132 mounted inside the recorder apparatus.

FIG. 4 shows a scent formulating and scent diffusing recorder apparatus according to a preferred embodiment of the present invention. The head assembly spraying unit 132a consists of an enclosed scent mixing chamber 144, with said scent mixing chamber 144 having a valve assembly consisting of a nipple 146, a bladder 146, a pressure valve 146, a piston 146, and interconnecting wires 146; an optional heating element 145; a piezoelectric transducer 142, a flexible thin mesh plate 141 material made of a plurality of materials such as metal or ceramic, with said mesh plate 141 having a multitude of small apertures, and interconnecting wires; an air pump 140 outfitted with a plastic syringe head with capillary action; and a rotating nozzle 132a head with and aperture and piston.

There is a scent formulating and scent diffusing recorder apparatus, multiple scent vessel assemblies 147 containing various scented oils, and in response to user's keyed input into application software module and under control of the microprocessor CPU 103 application software module 104 and apparatus controller interface 110a head assembly spraying unit scent mixing chamber 144 is moved left and right by belt 133 allowing assemblies 144 to mix scented oils for subsequent atomization by the piezoelectric transducer 142 and later collection of atomized scent oil droplets by the air pump for transport to the rotating spray nozzle 132a for diffusion on to recording papers 138.

Head ink cartridge 139 is installed on head ink assembly 139 and scent vessel cartridge 147 is installed on scent assemblies 147. Belt 133 is moved with a pulley sprocket 133 attached to carriage convey motor 149. Head ink assembly 139 sprays ink particles on recording papers 138's surface so that characters are recorded, and the head assembly spray unit's 132a rotating nozzle sprays mixed scented oil on recording papers 138's targeted pixel region so that the scented oil sinks into the recording papers 138. Additionally, scent assemblies 132a selectively spray various kinds of scented oils on recording papers 138 using said piezoelectric effect 142 in response to microprocessor 100 CPU 103 application software module 104 and apparatus controller interface 110a.

In a second preferred embedment (not shown) of the present invention, scent assemblies selectively spray various derivative mixed scented oils on recording papers using a thermal effect in response to microprocessor CPU, application software module, and apparatus controller interface.

In a preferred embodiment of the present invention, there is a scent formulating and scent diffusing recorder apparatus, a head ink assembly 139 slides on a rail 132 mounted inside the recorder, and is moved left and right by a belt 133 to form characters on sheets of recording papers 138. Scent vessel assemblies 132a are installed in recorder so as to selectively spray various scents on targeted regions of recording papers 138. Spray assemblies 132a selectively mix and spray various mixtures of scented oil on recording papers 138 using a piezoelectric or thermal effect in response to the microprocessor 100 CPU 103 application software module 104 and apparatus controller interface 110a. As recording papers 138 are fed into recorder, head ink assembly 139, connected to belt 133 and sliding on rail 132, is moved left and right so as to form characters within the recording region (a) of recording papers 138. Spray unit assemblies 132a, located over the targeted region of recording papers 138 scatter scented oils on recording papers 138.

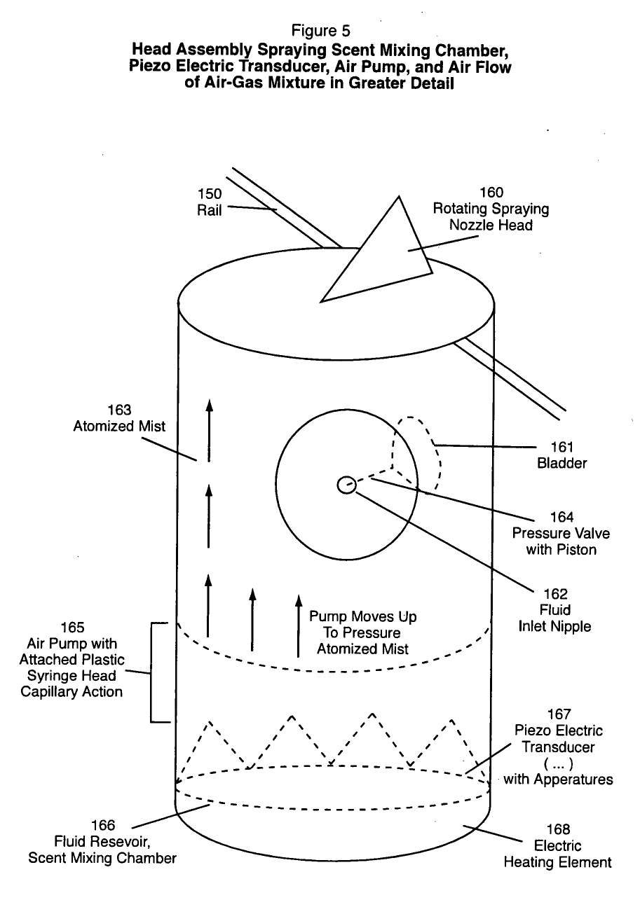

FIG. 5 shows the head assembly spraying unit scent mixing chamber 166 piezoelectric transducer 167 air pump 165 and air flow of an air-gas mixture 163 in greater detail (relative to FIG. 4) and shows the scent formulating and scent diffusing recorder apparatus, wherein said head assembly spraying unit 132a is under piezoelectric control for spraying scent onto addressable targeted areas of the recording papers 138 according to user mouse clicked keystrokes into a common commercially available microprocessor. FIG. 5 also shows the head assembly spraying unit according to claim 2, further comprising a reverse air path process, for supplying air in which no scent is mixed in said scent mixing chamber, in order to purge a residual scent therein.

As shown in FIG. 5 in a preferred embodiment of the present invention, once in the mixing chamber 166 predetermined amounts of one or more constituent scented oils are mixed, heated and where said oils are subjected to electrical current and vibrational energy by a piezoelectric transducer 167 for conversion of said oils to atomized vapor droplets 163. A more detailed review of the atomization process reveals that after predetermined amounts of one or more constituent scented oils are mixed and heated in the scent mixing chamber 166, the bottom side of the thin mesh plate 167 is in contact with the mixed scented liquids which are to be atomized on an “on-demand” basis wherein the mesh plate 167 and liquid is vibrated at ultrasonic frequencies to atomize the liquid as it passes through the plate into the diffusion chamber. In operation of the atomizer, alternating electrical voltages from an external source are applied through the leads to electrically conductive coatings on the upper and lower surfaces of the actuator 39a. This produces a piezoelectric effect in the actuator material whereby the actuator expands and contracts in radial directions. As a result, the diameter of the aperture holes increases and decreases in accordance with these alternating voltages. These changes in diameter are applied as radial forces on the membrane; and as a result, the flange region of the plate 167 flexes and pushes the domed center region up and down. This produces a pumping action on the liquid which is brought up against the underside of the center region of the mesh plate 167. As a result of the rapid ultrasonic vibrations, the liquid is forced upwardly through the perforations and is ejected from the upper surface of the membrane as a mist into the vicinity of the air pump.

Subsequently, the air pump 165, under control of the apparatus CPU 134 and outfitted with a plastic syringe head with capillary action collects said atomized oil vapor droplets 163 and by using air pressure, the air pump transfers said atomized oil vapor droplets 163 to the head assembly spraying unit 160 whereby a piston adds air to said atomized oil droplets and a rotating nozzle head 160 diffuses the oil droplet-air mixture 163, spraying the atomized oil droplet-air mixture 163 onto a sheet of recording paper 138 while the head assembly spraying unit rotating nozzle 160 is moved by a belt 133 during the recording process. After the rotating nozzle head 160 has sprayed the scent, the apparatus begins a purging process to rid the scent mixing chamber 166 of residual scent left by operation thereof. The apparatus CPU 134 commands the piston 164 in the scent mixing chamber valve assembly 164 to open the bladder 161 and nipple 162 for air intake into the scent mixing chamber 166 from the space surrounding the head assembly spraying unit 160, the lapsed time of said air in take being monitored by a clock in the apparatus and signaling to the apparatus CPU 134 to reverse the piston 164 and thereby close the bladder 161 and nipple 162, thus ceasing air intake into the scent mixing chamber 166. The apparatus CPU 134 then reverses the direction of aforementioned air pump 165, thus increasing air pressure in the scent mixing chamber 166, the lapsed time of said air pump 165 movement being monitored by a clock in the apparatus and signaling to the apparatus CPU 134 to move said piston 164 in the scent mixing chamber 166 valve assembly 164 to open the bladder 161 and nipple 162 for air exhale or purge outward from the scent mixing chamber 166 and into the space surrounding the head assembly spraying unit 160, the lapsed time of said air purge being monitored by a clock in the apparatus and signaling to the apparatus CPU 134 to reverse the piston 164 and thereby close the bladder 161 and nipple 162, thus ceasing air purge out of the scent mixing chamber.

In past piezoelectric ink jet systems, a principal problem is associated with the relative disparity in size between the piezoelectric transducer and the ink jet orifice. With recent advancements in piezoelectric transducer technology, the transducer is generally and substantially the same size as the orifice, thereby creating great design and functional flexibility with regard to either the minimum separation of the jets or the number of jets which can be used on a given recording head. Furthermore, piezoelectric transducers are now relatively in-expensive to produce and are amenable to many of the modern semiconductor fabrication techniques.

In a second preferred embodiment (not shown) of the present invention, once in the mixing chamber, predetermined amounts of one or more constituent scented oils are mixed, heated and where said oils are subjected to further thermal energy for conversion of said oils to vapor droplets by means of evaporation. Subsequently, an air pump outfitted with a plastic syringe head with capillary action collects said oil vapor droplets and by using air pressure, the air pump transfers said oil vapor droplets to the head assembly spraying unit, whereby a piston adds air to said oil droplets and a rotating nozzle head diffuses the oil droplet-air mixture, spraying the oil droplet-air mixture onto a sheet of recording paper while the head assembly spraying unit rotating nozzle is moved by a belt during the recording process.

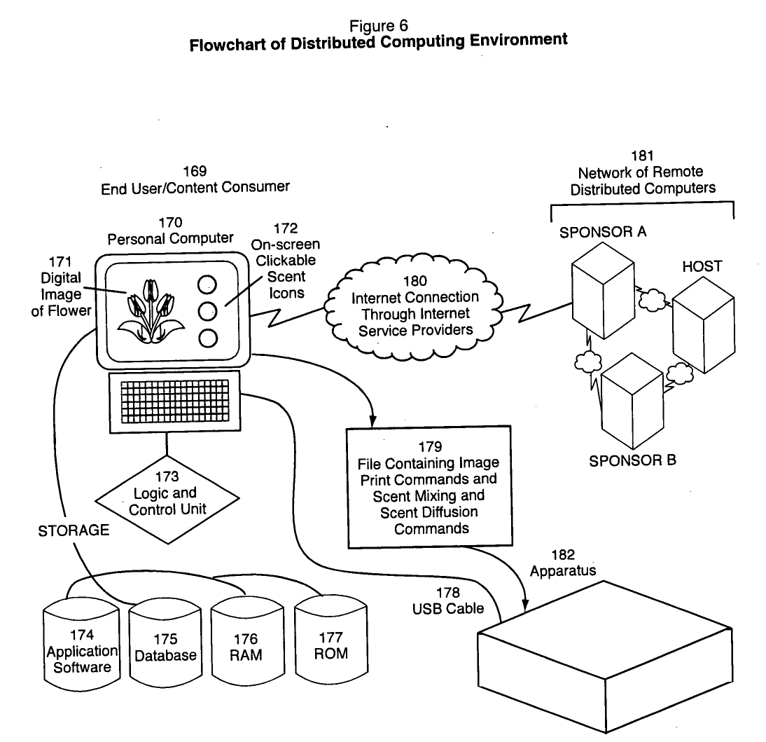

FIG. 6 is a flowchart of a distributed computing environment for the scent formulating and scent diffusing recording apparatus according to a preferred embodiment of the present invention;

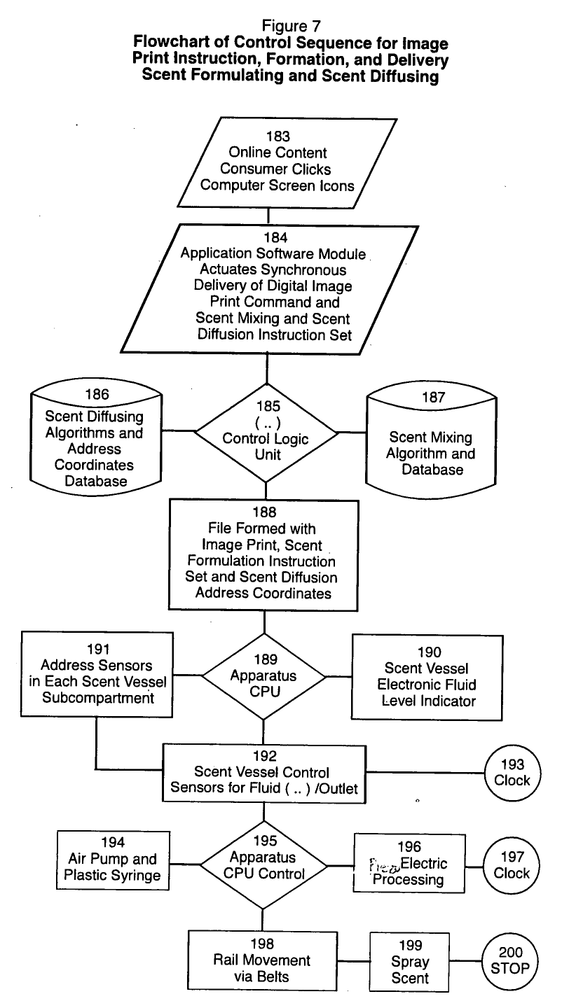

FIG. 7 is a flowchart of the control sequence for image recording instruction formation and delivery, and scent formulating and scent diffusing by the recording apparatus and identifies addressable targeted as areas that are spaces consisting of pixels inside recorded characters and recorded computer generated images, graphic art, line art, and text images according to user mouse clicked keystrokes into a common commercially available microprocessor 100. FIG. 7 also shows how scent is sprayed onto targeted areas of the recording papers 138 according to user mouse clicked keystrokes into a common commercially available microprocessor. FIG. 7 also shows the scent spraying recorder apparatus of claim 9, wherein said targeted areas are spaces consisting of pixels inside recorded characters and recorded computer generated images, graphic art, line art, and text images according to user mouse clicked keystrokes into a common commercially available microprocessor 100.

FIG. 7 shows a flowchart of the control sequence for image recording instruction formation and delivery, and scent formulating and scent diffusing by the recording apparatus according to a preferred embodiment of the present invention where the user initiates the process sequence by interacting with the application software module 104. As previously mentioned, the user is looking at and desiring to actuate the synchronous recording of a specific digital image 101 appearing on the screen of their microprocessor 100 the formulating of a specific scent associated with said digital image, and the diffusing of that specific scent associated with said digital image appearing on the screen of his microprocessor, with said microprocessor interacting with a remotely located computer (shown in FIG. 6) or a network of distributed remotely located computers (shown in FIG. 6) such as the world wide web, commonly referred to as the Internet, with said microprocessor 100 and remotely located computer or computers being outfitted with application software module 104 and its database 105, user's interaction 183 with microprocessor 100 resident application software module 104 and its database 105 causes subsequent simultaneous interaction with the logic and control unit of the remotely located computer(s) 181 and with the logic and control unit 103 of the user's said microprocessor 100, which in turn, causes an electronic signal to synchronously actuate 184 both an image recording command and scent formulating and scent diffusion commands, associated with the image 101, to be delivered to and accepted by the controller interface 112 and logic and control unit 134 and 185 located in the scent formulating and scent diffusing recording apparatus. It is understood that each logic and control unit consists of a series of separate functional elements including central processing unit (CPU), temporary random access memory (RAM) 135 also referred to as execution memory, and read only memory (ROM) 136 also referred to as program memory. It is further understood to those skilled in the art, that digital images include computer generated images, graphic art, line art, and text images and the like.

In a preferred embodiment of the present invention, there is provided an apparatus resident CPU 185 which receives a digital image from the microprocessor. In the preferred embodiment, the means to actuate the synchronous transfer 184 of both digital image recording commands and scent formulating and scent diffusion commands, associated with said digital image 101 from the microprocessor 100 CPU 103 and the application software module 104 to the apparatus controller interface 112 and logic and control unit 185 located in the scent formulating and scent diffusing recording apparatus, is achieved by virtue of user input 183 into application software module 104. Upon user input 183, application software module 104 creates in the microprocessor 100 CPU 103, saves in the microprocessor 100 random access memory (RAM )106, and subsequently transfers from the microprocessor RAM 106 to the apparatus' RAM 115 memory, an image file 188 containing said digital image 101, said digital image recording commands, said image specific scent formulation commands 187, and said image specific scent diffusion commands 186, with application software module 104 accessing look-up tables in its database 105 to identify, retrieve and save in said image file 188, previously stored data representative of the derivative scent formula, associated with the digital image 101, and the identity and amount and viscosity of such basic constituent scented oils to be mixed to achieve the derivative scent, and data with the location coordinates of where the digital image is to be recorded and location coordinates of where the scent is to be diffused relative to where the digital image is to be recorded. Among the data retrieved from the database, the digital scent diffusion commands 186 include addressable targeted area coordinates identifying spaces consisting of pixels inside recorded characters and recorded computer generated images, graphic art, line art, and text images where scent is to be sprayed, and the digital image recording commands include number of digital pixels which represents a continuous tone colored image, made form a spectrum of derivative colors using the base colors of Magenta, Cyan, Yellow, and Black.

Having successfully created and transferred digital image recording commands and scent mixing 187 and scent diffusion commands 186 from the microprocessor CPU 103 to the recorder apparatus controller interface 112, the apparatus controller interface 112 transfers said commands to the apparatus CPU 189 which begins a sequence of control commands to achieve scent mixing and scent diffusion.

Upon receipt of an electronic signal from the microprocessor CPU 189, application software module 104, and apparatus controller interface 112, and apparatus CPU 189 in a serial fashion, the spherical scent vessel 147 is rapidly rotated on its pivoting axis 127a by means of a motor 148, with said rotation stopping when a scent vessel-sub-compartment 126a bearing an address 191 that matches the identity of basic constituent scented oils, as predetermined by the application software module 104 database 105, has its nipple 130a and 192 positioned for direct connection to the head spray assembly unit's scent chamber intake valve 130b and 192 on the spraying unit assembly 130, the spraying unit assembly of which, under control of the apparatus' CPU 189, slides into position and then remains fixed during the impregnation process, whereby a precise amount of each database 105 selected scented constituent oil from each sub-divided scent vessel assembly is released and transported into the adjacent scent mixing chamber. To get the scented oil into the mixing chamber 160, the apparatus CPU 195 commands the pistons in both the scent vessel valve assembly 129a and the scent mixing chamber valve assembly 129b to open the respective bladders 129a and 129b and nipples 130a and 130b for scented oil intake 192 into the scent mixing chamber 160 from the scent vessel 122, the lapsed time of said scented oil in take being monitored by a clock 193 in the apparatus to meter the correct amount of fluid transfer based on predetermined fluid transfer rates, and signaling to the apparatus CPU 195 to reverse the piston 129a and 129b movements and thereby close the bladders 129a and 129b and nipples 130a and 130b of both the scent vessel and scent mixing chamber, thus ceasing scented oils intake into the scent mixing chamber 160.

Once in the mixing chamber 160, predetermined amounts of one or more constituent scented oils are mixed, by virtue of the apparatus CPU 195 commanding the motor 148 to move, thus driving the belts 198 which in turn move the entire head assembly spraying unit back and forth on the rail heated and where said oils are subjected to electrical current and vibrational energy 196 by an apparatus CPU 195 controlled piezoelectric transducer 196 for conversion of said oils to atomized vapor droplets.

Subsequently, the air pump 194, under control of the apparatus CPU 195 and outfitted with a plastic syringe head with capillary action collects said atomized oil vapor droplets and by using air pressure, the air pump 194 transfers said atomized oil vapor droplets to the head assembly spraying unit, whereby a piston adds air to said atomized oil droplets and a rotating nozzle head diffuses the oil droplet-air mixture 199, spraying the atomized oil droplet-air mixture 199 onto a sheet of recording paper 138 while the head assembly spraying unit rotating nozzle is moved by a belt during the recording process. When the recording and scent diffusion operations are to be carried out, apparatus CPU 195 moves ink head assembly 139 to recording papers 138's recording region where characters are to be formed, and recordings ink to on images selected by user on a application software selected regions of recording papers 138. Head assembly 46 sprays ink particles to form characters or graphics on recording papers 138. Subsequent to characters being recorded, apparatus CPU 195 moves the head assembly spraying unit to recording papers 138's recording region where scent is to be sprayed, and sprays scented oil recording papers on addressable targeted areas selected by application software 104 and 184, wherein said addressable targeted areas are spaces consisting of pixels inside recorded characters and recorded computer generated images, graphic art, line art, and text images on recording papers 138.

After the rotating nozzle head has sprayed the scent, the apparatus begins a purging process to rid the scent mixing chamber of residual scent left by operation thereof. The apparatus CPU 195 commands the piston in the scent mixing chamber 130 valve assembly to open the bladder 128b and nipple 130b for air intake into the scent mixing chamber 130 from the space surrounding the head assembly spraying unit, the lapsed time of said air in take being monitored by a clock 194 in the apparatus and signaling to the apparatus CPU 195 to reverse the piston 129b and thereby close the bladder 128b and nipple 130b, thus ceasing air intake into the scent mixing chamber 130. The apparatus CPU 195 then reverses the direction of aforementioned air pump 165, thus increasing air pressure in the scent mixing chamber 130 the lapsed time of said air pump movement being monitored by a clock 194 in the apparatus and signaling to the apparatus CPU 195 to move said piston 129b in the scent mixing chamber valve assembly to open the bladder 128b and nipple 130b for air exhale or purge outward from the scent mixing chamber 130 and into the space surrounding the head assembly spraying unit, the lapsed time of said air purge being monitored by a clock in the apparatus and signaling to the apparatus CPU 195 to reverse the piston and thereby close the bladder 128b and nipple 130b, thus ceasing air purge out of the scent mixing chamber.

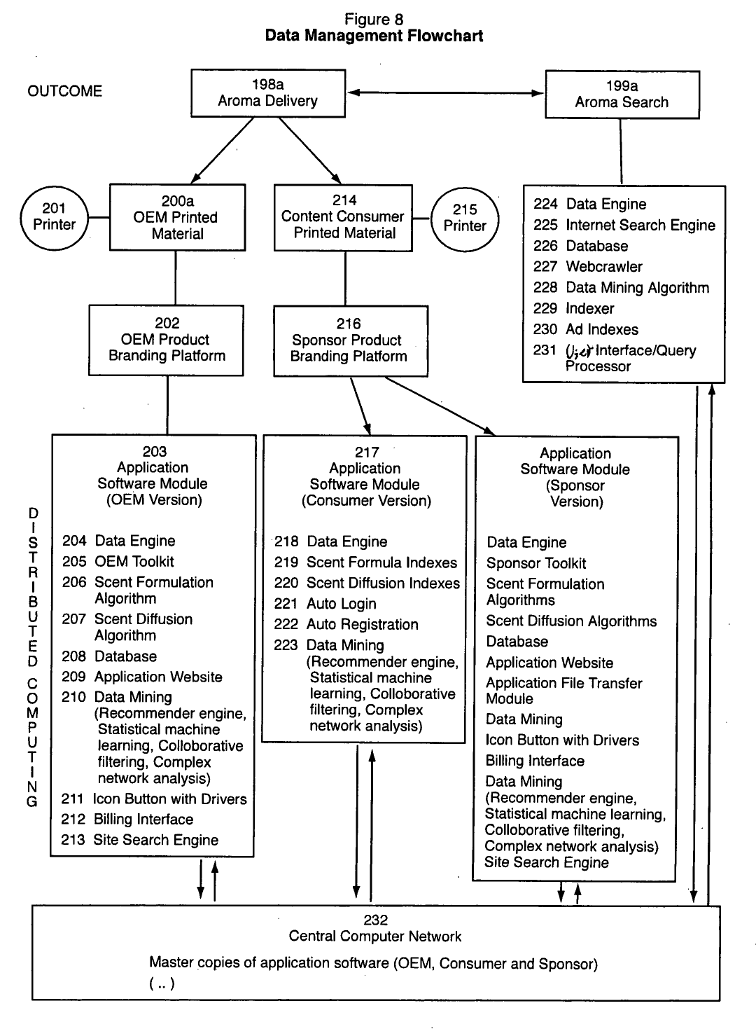

As shown in FIG. 8 “Data Management Flowchart”, there is a Data Management method, in the preferred embodiment comprising the components of;

(a) constructing and storing a master original database and two copies of said master original database. The master original database being accessible through a centrally located computer, a first copy of said master original database being accessed and manipulated via the computers of remotely located end users over a network of distributed computers such as the Internet, with manipulations, including custom scent formulas, categorization, etc . . . having separate storage and capable of being forwarded via said remotely located end user computers, and a second copy of said master original database being accessed and manipulated via the computers of remotely located hosting sponsors over a network of distributed computers such as the Internet, with manipulations, including custom scent formulas, categorization, etc . . . having separate storage and capable of being forwarded via said remotely located hosting sponsor computers. Each database contains information necessary for producing scents to be diffused, said databases including the positions where each original scent is contained, and the kind, constituent ratio, concentration and diffusion condition of the original scents to be combined in order to make a set scent, where each formulated scent is to be sprayed within targeted areas on recording papers, wherein said targeted areas are spaces consisting of pixels inside recorded characters and recorded computer generated images, graphic art, line art, and text images, and where images, associated with user selected scents, are to be recorded;

(b) constructing and storing a separate master original database, being accessed by a centrally located computer, containing remotely located end user profiles, including but not limited to user demographics such as gender, names, ages, ethnicity, education levels, annual incomes, geographic locations, e-mail addresses, etc . . . , and containing a history of previous user visits, sessions, and selected scents matched with, for example, and not limited to, associated sponsor product numbers, sponsor brand numbers, user demographics, and the like, for purposes of analyzing and predicting end user scent selection behavior, using a recommender engine consisting of algorithms for statistical machine learning, collaborative filters, and complex network based analysis. Also contained in this master original database is transaction counting mechanism that assigns and tracks a unique transaction number comprised of a user identification number, a sponsor brand number, and a sponsor product number, and a numeric suffix, with the numeric suffix being automatically incremented to account for the frequency of end user downloads of a particular scent and product combination;

(c) said algorithms comprised of mathematical models using, among other means, classification and regression trees (CART), multivariate adaptive regression splines (MARS), Box-Jenkins and Bayesian statistical modeling, predicting, and forecasting techniques;

(d) computer interface and mouse clicked key stroked commands using a common commercially available personal computer to enable remotely located end users to search said databases in step (b) and in step (c) to locate products, brands and the like that match user desired scents. The method of remote end user search disclosed herein is based on algorithms that determine relevance of various factors from data bits stored in a centrally located computer and ranks search results according to highest to least relevancy.

Having thus described the preferred embodiments in some detail it will be clear to those having ordinary skill in the art that obvious modifications could be made to the system without departing from the spirit of the invention. Such obvious modifications are within the inventive concept. It will be apparent that various modifications can be made in the particular scent formulating and scent diffusing apparatus described in detail above and shown in the drawings within the scope of the invention. For example, the size, shape, and configuration of the components can be changed to meet specific requirements. The present invention presents methods, systems and an apparatus to deliver scent in a digital format. It is therefore possible for the invention to be applied to end terminal scent diffusing digital devices such as recorders, computers, mobile phones, terrestrial land-line based phones, digital televisions, compact disc (CD) players, digital video disc (DVD) players, MP3 players, digital and satellite radio receivers, medical equipment, including but not limited to respirators, the ventilation systems of buildings, automobiles, ships, aircraft, and railway, slot machines, theatre systems, refrigerators, game machines, amusement park rides, clothes washing machines, clothes drying machines, dishwashing machines, and vacuum cleaning machines, with the present invention providing scent diffusion into the atmosphere in close proximity to such digital devices. Also, the components can be disposed in various orientations provided the operation and functioning of the apparatus are not deleteriously affected. In addition, the components can be arranged differently. Various changes may be made in shape, size and arrangement of parts. For example, equivalent elements or materials may be substituted for those illustrated and described herein. Parts may be reversed, and certain features of the invention may be utilized independently of the use of other features, all as would be apparent to one skilled in the art after having the benefit of having this description of the invention. It is not intended that the words used to describe the invention nor the drawings be limiting on the invention, rather the only limitations placed are those in the appended claims.

What is claimed is:

1. A scent formulating and scent diffusing apparatus for synchronously mixing one or more scents, spraying one or more scents, and recording a digital image upon electronic signal demand, with said signaling mechanism either located directly within a common commercially available microprocessor, or over a network of distributed computers such as the world wide web, commonly referred to as the Internet, comprising:
a controller, commonly found in commercially available microprocessors, separate from the apparatus, generating a signal on demand from the controller, an input of the apparatus accepting the signal;
a graphics card and corresponding on-screen icon image, commonly found in commercially available microprocessors, when actuated, synchronously transferring both digital image recording commands and digital scent diffusion commands to
a computer application software module which, in turn, formats, synchronizes and transfers digital image recording commands and digital scent formulation and digital scent diffusion commands to the apparatus upon demand as a function of the signal;

2. A scent formulating and scent diffusing apparatus comprising:
a belt;
a rail within the recorder to be moved by said belt;
a head assembly spraying unit sliding on said rail within the apparatus to spray scent on recording subject; and
one or more scent vessel assemblies, each capable of being sub-divided with compartmentalized walls, located in the apparatus, spaced apart from the head assembly spraying unit and positioned to selectively spray scent on the recording subject in a region to be recorded on.

3. A scent formulating and scent diffusing apparatus comprising:
a belt;
a rail within the recorder to be moved by said belt;
an recording head assemlbly sliding on said rail within the apparatus to record on recording subject; and
one or more scent vessel assemblies located in the apparatus, spaced apart from the head assembly spraying unit and positioned to selectively spray scent on the recording subject in a region to be recorded on.

4. A rail mounted inside said apparatus;
a head assembly spraying unit for receiving and mixing precise amounts of one or more scented fragrant oils to produce a scent ,and, subsequently, diffusing said scent, with said head assembly spraying unit, sliding, upon demand as a function of the signal, on said rail mounted inside the apparatus;
one ore more one scent vessel assemblies, each capable of being sub-divided with compartmentalized walls, said one or more scent vessel assemblies, wherein stored are a plurality of individual scented oils, attached to the head assembly, each of said one or more scent vessel assemblies containing fragrant oil; said one or more scent vessel assemblies transferring scent to the scent chamber and subsequently to the head assembly spraying unit for spraying scent onto sheets of recording subject as said head assembly rotating nozzle is moved to recording on the recording subject:
an electronic address to identify sub-divided scent vessel assembly locations where scented fragrant oils are stored;
an electronic benchmark sensor located inside each sub-divided scent vessel assembly for purposes of indicating remaining levels of scented fragrant fluid inside the sub-divided scent vessel assemblies; and
a bladder, nipple, pressure valve, piston and tube, each connected to each sub-divided scent vessel assembly for purposes of releasing and transporting precise amounts of scented fragrant oil from each sub-divided scent vessel assembly to the head assembly spraying unit;

5. The scent formulating and scent diffusing apparatus according to claim 1, wherein said head assembly spraying unit is under piezoelectric control for spraying scent onto addressable targeted areas of the recording subject according to user mouse clicked keystrokes into a common commercially available microprocessor;

6. The head assembly spraying unit according to claim 2, further comprising a reverse air path process, for supplying air in which no scent is mixed in said scent mixing chamber, in order to purge a residual scent therein.

7. A Data Management method, comprising the components of;
(a) constructing and storing a master original database and two copies of said master original database. The master original database being accessible through a centrally located computer, a first copy of said master original database being accessed and manipulated via the computers of remotely located end users over a network of distributed computers such as the Internet, with manipulations, including custom scents, categorization, etc . . . having separate storage and capable of being forwarded via said remotely located end user computers, and a second copy of said master original database being accessed and manipulated via the computers of remotely located hosting sponsors over a network of distributed computers such as the Internet, with manipulations, including custom scents, categorization, etc . . . having separate storage and capable of being forwarded via said remotely located hosting sponsor computers.
(b) constructing and storing a separate master original database, being accessed by a centrally located computer, containing remotely located end user profiles.
(c) algorithms comprised of mathematical models using, among other means, Box-Jenkins and Bayesian statistical modeling, predicting, and forecasting techniques;
(d) computer interface and mouse clicked key stroked commands using a common commercially available microprocessor to enable remotely located end users to search said databases.

8. A scent formulation method, comprising the steps of;
(a) storing a plurality of individual original scents;
(b) reading said information concerning said scent to be diffused from said database constructed according to claim 7 (a); and
(c) mixing and spraying corresponding one or more individual original scents according to said information read in said step 8 (b).

9. The scent diffusion method according to claim 8, wherein at least one of the individual original scents of said step (a) is contained in each of a plurality of scent spraying units.

10. The scent diffusion method according to claim 8, wherein the original scents of said step (a) are contained in a plurality of scent cartridges.

11. The scent diffusion method according to claim 10, wherein said diffusion condition comprises a diffusion point in time, a diffusion duration time and a diffusion intensity.

12. The scent diffusion method according to claim 9 wherein said diffusion condition comprises a diffusion point in time, a diffusion duration time and a diffusion intensity.

13. The scent diffusion method according to claim 12, wherein said step (d) comprises:
(d1) driving a corresponding scent spraying unit and evaporating the original scents as much as a predetermined amount;
(d2) uniformly mixing the scent evaporated in the corresponding scent spraying unit in said step (d1) with air, and producing a mixed gas to thereby produce a scent to be diffused; and
(d3) pressing the scent made in step (d2) according to a pressure difference, to then be transferred to recording subject located in the apparatus

14. A scent diffusion method according to claim 13, wherein the scent spraying unit is driven in said step (d1), so that a saturation rate of the evaporated original scent in each scent spraying unit is the same.

15. The scent diffusion method according to claim 14, wherein the evaporation rate depending upon a temperature of the original scent is differently set according to the position where each original scent is contained.

16. The scent diffusion method according to claim 13, wherein said step (d2) comprises the sub-steps of:
(d2a) checking the diffusion condition, and supplying the air into the scent spraying unit containing the original scent to form the scent to be diffused, to thereby be mixed with the evaporated scent;
(d2b) discharging the scent mixed with the air from the air pump; and
(d2c) uniformly mixing the air mixture gases discharged from said air pump.

17. The scent diffusion method according to claim 11, wherein said step (d) comprises the sub-steps of:
(d1) individually spraying corresponding original scents as much as a predetermined amount;
(d2) evaporating the corresponding original scents sprayed in said step (d1); and
(d3) discharging a mixed gas of the evaporated scent and the air.

18. The collective claims 1 through 18 consecutively and associated means herein for methods, systems, and apparatus, constitute the present invention of creating and transmitting aroma delivery instructions over the Internet, herein referred to as aroma casting, aroma streaming, or online scent management.
